(19) United States
(12) Patent Application Publication
(10) Pub. No.: US 2025/0288283 A1
(43) Pub. Date: Sep. 18, 2025

(54) Body Fluid Collection Device

(71) Applicant: Blackfly Investments, LLC, DBA Molecular Testing Labs, Vancouver, WA (US)

(72) Inventors: Charles Nokes, Portland, OR (US); Peter Flynn, Camas, WA (US); Adam Blackwell, Vancouver, WA (US)

(21) Appl. No.: 18/603,536

(22) Filed: Mar. 13, 2024

(51) Int. Cl. A61B 10/00

(52) U.S. Cl. CPC A61B 10/007

(57) ABSTRACT

A diagnostic testing aid for collecting body fluids for medical and health care industries. The collection device may comprise a conical envelope having a first end configured to be a closed end and a second end configured to be an open end. The conical envelope forms a cavity configured to hold liquid, such as urine from a urine stream. As a collection procedural step, the collection device may be set on a table upright with the use of a rigid member flexibly coupled to the conical envelope along a first joint which may be away from the conical envelope to provide support the conical envelope in the upright position. Then, when a shipping container is prepared, the collection device aids in pouring a sample into the shipping container using a crease and a tip in the conical envelope.

### BACKGROUND

As techniques and methodologies for medical testing and diagnostics advance, more consumers are turning to “at-home” collection kits that may involve collecting a urine sample using a collection device without the assistance of a physician/medical staff. In a typical procedure, a patient will induce urine flow into a dedicated collection medium, such as a cup or other receptacle. As is known in the profession, urine collection cups may be used to capture a sample and then the sample cup is packaged for mailing or transit to a remote testing facility. After testing the “at home” collected sample, results of testing or diagnostics may then be communicated to the patient using standard confidentiality protocols.

However, the standard urine collection cup form is difficult to ship, both before sample collection and after sample collection. A standard urine collection cup is a plastic round or cylindrical cup that has rigid walls. The size of the collection device before and after collection remains uniform and bulky. This presents issues with respect to shipping and handling as the container itself, whether containing patient sample or not, is a three-dimensional object that requires a bulky shipping container for the sample cup to be sent for testing.

### BRIEF DESCRIPTION OF THE DRAWINGS

Embodiments of the subject matter disclosed herein in accordance with the present disclosure will be described with reference to the drawings, in which:

FIG. 1 is isometric diagram of a body-fluid collection device according to an embodiment of the subject matter disclosed herein;

FIG. 2 is another isometric diagram showing a top view of the body-fluid collection device of FIG. 1 according to an embodiment of the subject matter disclosed herein;

FIG. 3 is a view of the body-fluid collection device of FIG. 1 when laid flat is a disassembled state so as to show the portion more clearly according to an embodiment of the subject matter disclosed herein; and

FIG. 4 is a diagram of a human using the body-fluid collection device of FIG. 1 according to an embodiment of the subject matter disclosed herein.

Note that the same numbers are used throughout the disclosure and figures to reference like components and features.

### DETAILED DESCRIPTION

The subject matter of embodiments disclosed herein is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

Embodiments will be described more fully hereinafter with reference to the accompanying drawings, which form a part hereof, and which show, by way of illustration, exemplary embodiments by which the devices described herein may be practiced. These devices may, however, be embodied in many different forms and should not be construed as limited to the embodiments set forth herein; rather, these embodiments are provided so that this disclosure will satisfy the statutory requirements and convey the scope of the subject matter to those skilled in the art.

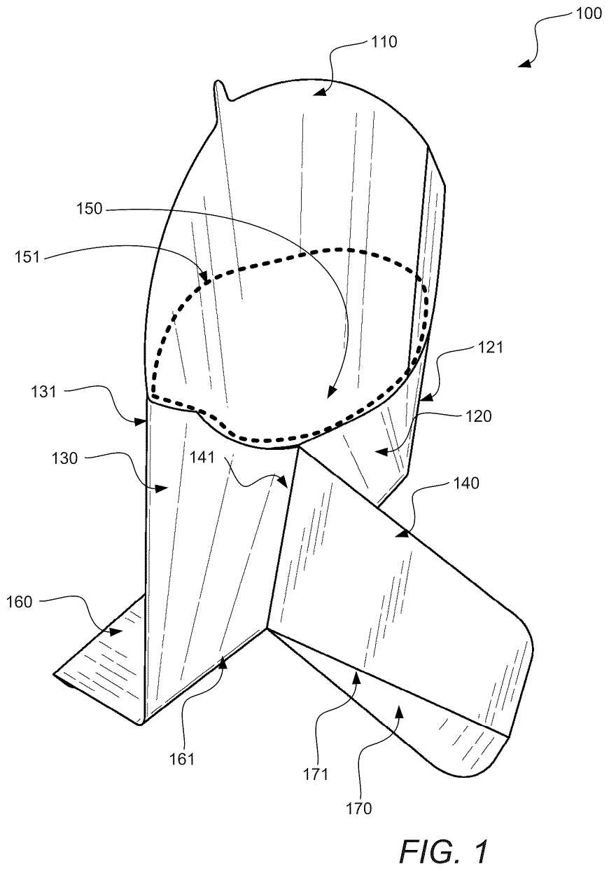

By way of an overview, the systems and devices discussed herein are directed to diagnostic testing aids for collecting body fluids for medical and health care industries when the collection is remotely accomplished. In an embodiment, a collection device may comprise a conical envelope having a first end configured to be a closed end and a second end configured to be an open end. The conical envelope forms a cavity configured to hold liquid, such as urine from a urine stream. As a remote collection procedural step, the collection device may be set on a table upright with the use of a rigid member flexibly coupled to the conical envelope along a first joint which may be away from the conical envelope to provide support the conical envelope in the upright position. Then, when a shipping container is prepared, the collection device aids in pouring a sample into the shipping container using a crease and a tip in the conical envelope. In this manner, additional equipment, such as pipettes, are not needed to prepare a sample for shipping in a proper shipping container. These and other aspects are discussed below with respect to FIGS. 1-4.

FIG. 1 is an isometric diagram of a body-fluid collection device 100 according to an embodiment of the subject matter disclosed herein. The body-fluid collection device 100, sometimes called a “P-Catch,” improves a process of body-fluid collection (e.g., capturing urine is one example) for patients that may benefit from remote health care and diagnostics. Generally speaking, the body-fluid collection device 100 is an envelope-like receptacle that can be conical in shape when deployed for the purpose of collecting liquid samples from patients, such as urine. Once the liquid is captured, the body-fluid collection device 100 may be then manipulated to more easily transfer the collected liquid into a more robust, yet small and narrow) transport tube, as needed.

The collection device 100, in the embodiment of FIG. 1 includes three rigid members 110, 120, and 130 that are assembled in a manner that lays flat when not is use (e.g., for initial manufacturing and transport) and can be manipulated into a receiving position having an orifice 151 (shown in FIG. 1 as an imaginary dotted line) over a cavity 150 for collecting liquid. In FIG. 1, the three members are shown as a first rigid member 110, a second rigid member 120, and third rigid member 130. FIG. 1 shows the collection device 100 manipulated into a receiving position such that the second rigid member 120 remains flexibly coupled (e.g., able to be rotated away from a generally flat position) to the first rigid member 110 along a first joint 121. Similarly, the third rigid member 130 remains flexibly coupled to the first rigid member 110 along a second joint 131. Finally, the second rigid member 120 and the third rigid member 130 remain flexibly coupled to each other along a third joint 141.

Each of the rigid members 110, 120, and 130 are also attached at the base of the collection device along a base joint 161. That is, each member 110, 120, and 130 is a substantially rectangular-shape and coupled at a base joint 161 along the bottom side of each member. In this manner, the members 110, 120, and 130 (that are not coupled to each other at respective topsides) may be pushed open, like a conical envelope. As each member 110, 120, and 130 remains uncoupled at the top of each substantially rectangular portion such that the orifice 151 may be formed when maneuvered to the open, receiving position to form the receiving cavity 150. A base support member 160 may be rotatably coupled to the three rigid members 110, 120, and 130 at the base joint as well and can be maneuvered to support the collection device 100 in an upright position when the base support member is rotated to a generally horizontal position (with respect to the three rigid members 110, 120, and 130).

The collection device 100 also includes at least one additional rigid member 140 used to provide support to maintain an upright receiving position when unfolded. Thus, in this embodiment, the maneuverable stand member 140 is rotatably coupled to the second and third members at the joint 141. In other embodiments not shown, there may be more than one rigid stand member 140 that may be maneuvered to support the collection device 100 in an upright receiving position. For example, an embodiment may have a first rigid stand member rotatably attached to the second rigid member 120 near the joint 121 as well as a second rigid stand member rotatably attached to the third rigid member 130 near the joint 131. In other embodiments not shown, the collection device may have more than two rigid stand members configured to be rotated away from one or more of the three rigid members 110, 120, and 130. In any embodiment with rigid stand members 140, each rigid stand member 140 may include its own respective rotatable flat portion 170 that may be folded at joint 171 to be horizontal and match the base support member 160.

As discussed above, the collection device 100 is designed to lay flat when manufactured and shipped (i.e., a folded position). When readying for use, a person may manipulate the collection device 100 by spreading out the first member 110, the second member 120, the third member 130 to form an open cavity for collecting body fluid (i.e., an unfolded position or an upright position).

The collection device 100 is an improvement to a process for collecting body fluid because the collection device 100 is easier for patients to capture urine in stream, especially those with female anatomy. Conventional collection devices may be a collapsible cup or a collection tube having a small opening. Using the body-fluid collection device 100 as an intermediary allows for transfer into secondary containers (e.g., container better suited for shipping or transport) easier without need for a pipette, or the like. Additionally, the collection device 100 holds a larger volume of liquid and is assembled in one piece providing a high level of stability (even post collection). Further, the collection device 100 is disposable, biodegradable and contains no plastic to be more environmentally friendly. Further yet, the manufacturing is simple as it involves a single-component construction process wherein the flat manufacture lends itself well to shipping in a flat envelope.

The collection device 100 includes all members made from a liquid resistant material. In other embodiments, the rigid members are made from water-tight and water-proof materials that remain biodegradable. In some embodiments, not shown, the cavity may further include a fluid-tight, lining made from a plastic material attached to inner walls of the three rigid members, though this may not be preferred if the collection device is to remain biodegradable. In further embodiments, the collection device 100 may include an inner lining further that is a sealable and resealable bladder having an opening at a top of the bladder positioned adjacent to the orifice. In such a bladder-filled interior, the collection device 100 may also be the shipping container once filled with a desired body fluid for medical or diagnostic reasons.

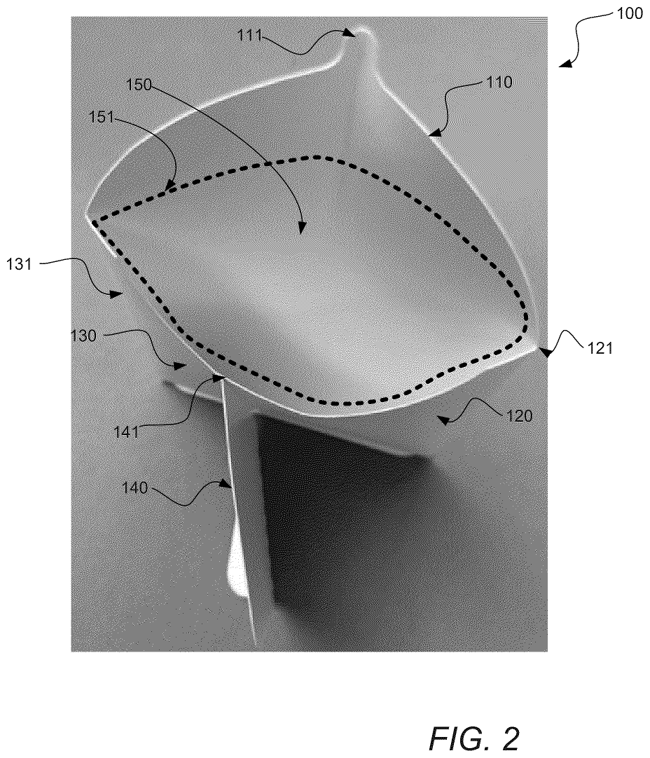

FIG. 2 is another isometric diagram showing a top view of the body-fluid collection device 100 of FIG. 1 according to an embodiment of the subject matter disclosed herein. In this view, one can see the same elements as discussed above with respect to FIG. 1. Namely, the collection device 100 is shown here with three rigid members 110, 120, and 130 that can be manipulated into a receiving position (as shown here) having an orifice 151 (shown in FIG. 2 as an imaginary dotted line) over a cavity 150 for collecting liquid. FIG. 2 shows the second rigid member 120 flexibly coupled (e.g., able to be rotated away from a generally flat position) to the first rigid member 110 along a first joint 121. Similarly, the third rigid member 130 is flexibly coupled to the first rigid member 110 along a second joint 131. Finally, the second rigid member 120 and the third rigid member 130 are flexibly coupled to each other along a third joint 141.

Each of the rigid members 110, 120, and 130 are also attached at the base of the collection device such that the rigid members 110, 120, and 130 may be pushed open, like a conical envelope. As each member 110, 120, and 130 remains uncoupled at the top of each substantially rectangular portion such that the orifice 151 may be formed when maneuvered to the open, receiving position to form the receiving cavity 150. The collection device 100 also includes at least one additional rigid member 140 used to provide support to maintain an upright receiving position when unfolded. Thus, in this embodiment, the maneuverable stand member 140 is rotatably coupled to the second and third members at the joint 141. When fluid is collected in the cavity, a tip 111 may be used to assist with pouring liquid out of the collection device (e.g., into a shipping container, as but one example).

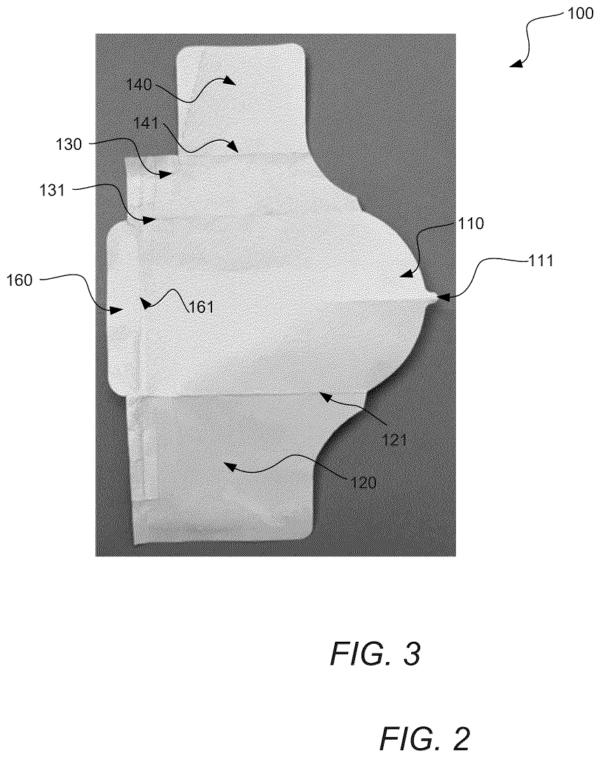

FIG. 3 is a view of the body-fluid collection device of FIG. 1 when laid flat is a disassembled state so as to show the portion more clearly according to an embodiment of the subject matter disclosed herein. In this view, one can see the same elements as discussed above with respect to FIGS. 1 and 2. Namely, the collection device 100 is shown here, in a disassembled position, with three rigid members 110, 120, and 130. FIG. 2 shows the second rigid member 120 still flexibly coupled to the first rigid member 110 along a first joint 121. Similarly, the third rigid member 130 is still shown flexibly coupled to the first rigid member 110 along a second joint 131. However, the second rigid member 120 and the third rigid member 130 have been separated for the purpose of this illustration.

Each of the rigid members 110, 120, and 130 are also shown here as being unattached at the base of the collection device 100 again for the purposes of illustration. The collection device 100 also includes at least one additional rigid member 140 used to provide support to maintain an upright receiving position when deployed. Thus, in this view, the maneuverable stand member 140 is rotatably coupled to the third member 130 at the joint 141. Also shown here, the base support member 160 is coupled to the first member 110 and joint 161. The tip 111 may be used to assist with pouring liquid out of the collection device.

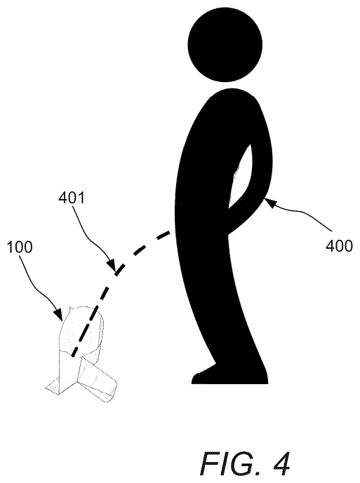

FIG. 4 is a diagram of a human 400 using the body-fluid collection device of FIG. 1 according to an embodiment of the subject matter disclosed herein. In FIG. 4, one can see that the human may generate a urine stream 401 to be collected in the collection device without holding onto the collection device 100 because it may sit upright. Alternatively, the orifice is large enough to aid in the collection of liquid from a urine stream 401 when one is strategically holding the collection device 100 without missing or experiencing a misfire onto one's hands (or worse).

The use of the terms “a” and “an” and “the” and similar referents in the specification and in the following claims are to be construed to cover both the singular and the plural, unless otherwise indicated herein or clearly contradicted by context. The terms “having,” “including,” “containing” and similar referents in the specification and in the following claims are to be construed as open-ended terms (e.g., meaning “including, but not limited to,”) unless otherwise noted. Recitation of ranges of values herein are merely indented to serve as a shorthand method of referring individually to each separate value inclusively falling within the range, unless otherwise indicated herein, and each separate value is incorporated into the specification as if it were individually recited herein. All methods described herein can be performed in any suitable order unless otherwise indicated herein or clearly contradicted by context. The use of any and all examples, or exemplary language (e.g., “such as”) provided herein, is intended merely to better illuminate embodiments and does not pose a limitation to the scope of the disclosure unless otherwise claimed. No language in the specification should be construed as indicating any non-claimed element as essential to each embodiment of the present disclosure.

Different arrangements of the components depicted in the drawings or described above, as well as components and steps not shown or described are possible. Similarly, some features and sub-combinations are useful and may be employed without reference to other features and sub-combinations. Embodiments have been described for illustrative and not restrictive purposes, and alternative embodiments will become apparent to readers of this patent. Accordingly, the present subject matter is not limited to the embodiments described above or depicted in the drawings, and various embodiments and modifications can be made without departing from the scope of the claims below.

That which is claimed is:

1. A collection device for collecting bodily fluid samples for diagnostics, comprising:
a first rigid member;
a second rigid member flexibly coupled to the first rigid member along a first joint;
a third rigid member flexibly coupled to the first rigid member along a second joint and flexibly coupled to the second rigid member along a third joint;
at least one maneuverable stand member rotatably coupled to the first member;
wherein the first member, the second member, the third member and the stand member are configured to lay flat when is a folded position; and
wherein the first member, the second member, the third member are configured to form an open cavity for collecting body fluid when is a receiving position such that the stand member is configured to be rotated to provide support for the first member, the second member, the third member to be positioned in in an upright position with an orifice exposing the cavity at a top side of the device the first member, the second member, the third member.

2. The collection device of claim 1, wherein the first rigid member, the second rigid member, the third rigid member are contiguous with each other at a respective bottom side of each of the first rigid member, the second rigid member, the third rigid member.

3. The collection device of claim 1, wherein the first rigid member, the second rigid member, and the third rigid member comprise a fluid resistant material.

4. The collection device of claim 1, wherein cavity further comprises a plastic, fluid- tight, lining.

5. The collection device of claim 4, wherein the lining further comprises a bladder having a resealable opening at a top of the bladder positioned adjacent to the orifice.

6. The collection device of claim 4, wherein the lining is attached to inside walls of the first rigid member, the second rigid member and the third rigid member that form the cavity when manipulated to be in the receiving position.

7. The collection device of claim 1, further comprising at least one second maneuverable stand member rotatably coupled to the first rigid member configured to be rotated into to provide support for the first rigid member, the second rigid member, the third rigid member to be positioned in in an upright position.

8. The collection device of claim 1, wherein the first rigid member, the second rigid member, and the third rigid member comprise substantially rectangular members.

9. The collection device of claim 1, wherein the first rigid ember comprises a rounded edge having a tip configured to aid in pouring liquid form the cavity.

10. A diagnostic aid device for collecting a urine sample, comprising:
a conical envelope having a first end configured to be a closed end and a second end configured to be an open end, the conical envelope forming a cavity configured to hold liquid; and
a rigid member flexibly coupled to the conical envelope along a first joint and configured to rotate away from the conical envelope to provide support the conical envelope in an upright position to expose the cavity.

11. The diagnostic aid device of claim 10, wherein the conical envelope further comprises a first rigid member, a second rigid member, the third rigid member contiguous with each other at a respective bottom side of each of the first rigid member, the second rigid member, the third rigid member.

12. The diagnostic aid device of claim 10, wherein the conical envelope comprises a fluid resistant material.

13. The diagnostic aid device of claim 10, wherein the conical envelope comprises a water-proof material.

14. The diagnostic aid device of claim 10, further comprising at least one second rigid member flexibly coupled to the conical envelope along a second joint and configured to rotate away from the conical envelope to provide support the conical envelope in in an upright position to expose a cavity for holding urine collected from a urine stream.

15. The diagnostic aid device of claim 1, wherein the rigid member further comprises a folding base portion configured to further support the conical envelope when sitting in an upright position.

16. The diagnostic aid device of claim 1, wherein the conical envelope further comprises a rounded edge having a tip configured to aid in pouring liquid form the cavity.

17. The diagnostic aid device of claim 1, wherein the rigid member further comprises a folding base portion configured to further support the conical envelope when sitting in an upright position.

18. The diagnostic aid device of claim 1, further comprising a base member coupled to the closed end of the conical envelope.

19. A fluid collection device, comprising:
a first member;
a second member flexibly coupled to the first member along a first joint;
a third member flexibly coupled to the first rigid member along a second joint and flexibly coupled to the second member along a third joint;
a plurality of maneuverable stand members each respectively coupled one of the first, second, or third members;
wherein the first member, the second member, the third member and the stand member are configured to lay flat when is a folded position; and
wherein the first member, the second member, and the third member are configured to form an open cavity for collecting body fluid when in a receiving position.

20. The collection device of claim 1, wherein the cavity is configured to collect urine from a urine stream.
